(19) United States
(12) Patent Application Publication
(10) Pub. No.: US 2025/0258153 A1
(43) Pub. Date: Aug. 14, 2025

(54) METHOD FOR PREDICTING HEAVY METAL ACCUMULATION IN SOIL BASED ON EMISSION INVENTORY AND RECEPTOR MODEL

(71) Applicant: KUNMING UNIVERSITY OF SCIENCE AND TECHNOLOGY, Kunming (CN)

(72) Inventors: Peng DONG, Kunming (CN); Chenchen LI, Kunming (CN); Qi MENG, Kunming (CN); Yingjie ZHANG, Kunming (CN); Wanquan YU, Kunming (CN); Jin YAN, Kunming (CN); Hanjing YU, Kunming (CN)

(21) Appl. No.: 19/121,559

(22) PCT Filed: Nov. 29, 2024
(86) PCT No.: PCT/CN2024/135433

§ 371 Date: Apr. 16, 2025

(30) Foreign Application Priority Data
Jan. 10, 2024 (CN) 202410036627.6

(51) Int. Cl. G01N 33/24; G06Q 10/04; G06Q 50/02

(52) U.S. Cl. CPC G01N 33/24; G06Q 10/04; G06Q 50/02

(57) ABSTRACT

The present application relates to the technical field of the treatment of heavy metal pollution, and in particular, to a method for predicting heavy metal accumulation in soil based on an emission inventory and a receptor model. Atmospheric dust fall, irrigation water, a fertilizer, and a pesticide are used as heavy metal input flux sources of farmland soil, and surface runoff and a crop are used as output fluxes. A heavy metal pollutant input-output flux inventory is established to specify a dynamic equilibrium relationship of heavy metal accumulation in soil, and soil samples are collected and monitored continuously, which provides important help for determining a migration equilibrium of heavy metals in a farmland region affected by a nonferrous metal dressing and smelting slag yard and a source of soil heavy metal pollution, thereby providing theoretical guidance for subsequent prevention and accurate control of heavy metals in soil.

### CROSS REFERENCE TO RELATED APPLICATION

This patent application is a national stage application of International Patent Application No. PCT/CN2024/135433, filed on Nov. 29, 2024, which claims the benefit and priority of Chinese Patent Application No. 202410036627.6 filed with the China National Intellectual Property Administration on Jan. 10, 2024, and entitled “METHOD FOR PREDICTING HEAVY METAL ACCUMULATION IN SOIL BASED ON EMISSION INVENTORY AND RECEPTOR MODEL”, the disclosure of which is incorporated by reference herein in its entirety as part of the present application.

### TECHNICAL FIELD

The present application relates to the technical field of the treatment of heavy metal pollution, and in particular, to a method for predicting heavy metal accumulation in soil based on an emission inventory and a receptor model.

### BACKGROUND

A heavy metal content in soil is mainly contributed by two input sources of a soil parent material and a human factor. Ultrahigh accumulation of heavy metals in soil may affect the soil ecosystem environment, and the heavy metals may be accumulated in the human body environment through the food chain and finally·harm the human health.

Main soil pollutants include lead, zinc, arsenic, copper, and some organic pollutants. At present, although scholars can obtain detailed soil pollution conditions, they may not know the source input of pollutants and predict the heavy metal accumulation in soil. In addition, there is no any technique involving a quantitative relationship between the source input of pollutants and the prediction of the heavy metal accumulation in soil at present. Therefore, adopting a reasonable and effective calculation method to predict the heavy metal pollution accumulation in soil and quantitatively identifying the source of the heavy metal pollution have great guidance significance for accurate remediation and control of farmland soil.

Identifying source categories and contribution rates of heavy metals in soil according to a correlation between contents of the heavy metals in soil and pollution features is called source apportionment. Three methods are usually adopted to study source apportionment: a source inventory method, a receptor model method, and a diffusion model method. The source inventory method involves estimating emission fluxes of different sources according to emission levels of different pollution sources. Such a calculation method is simple and clear, but cannot count various pollution sources systematically and accurately because it is hard to collect historical data. The diffusion model method involves estimating contributions of different source categories to heavy metals in soil according to a heavy metal emission pollutant inventory and a pollutant transport process. However, since the heavy metal emission and migration processes are complex and may be counted with difficulty, it is hard to establish a direct relationship between a pollution source and soil through pollutants. Therefore, the use of this method has a great limitation.

In order to solve the problems of the previous two methods, the receptor model method is carried out with a heavy metal having a source indication function for a soil sample and a pollution source, and involves qualitatively identifying a pollution source and quantitatively calculating contributions of different pollution sources to a heavy metal content in soil. Existing receptor models mainly include a positive matrix factorization (PMF) method, a UNMIX model, absolute principal component analysis/multiple linear regression (APCS-mLR), an isotope ratio method, a finite mixture distribution model (FMDM) and random forest in an advanced statistical algorithm. In these models, since a factor matrix is limited to be a non-negative value, PMF analysis may obtain a more significant factor and thus can be widely used. This model was applied to the source apportionment of atmospheric particle matter at the earliest. In recent years, some scholars have gradually attempted to apply this model to soil and sediments. Compared with the previous two methods, the receptor model method does not need too much historical information and is to directly measure a heavy metal content in soil by ignoring the heavy metal transport process. Therefore, the receptor model method is widely used in terms of the source apportionment of heavy metals in soil. Although the receptor model may monitor the heavy metal content in soil, it cannot dynamically describe a heavy metal change in soil at time and space levels.

Establishing an input and output flux emission inventory is an advantageous method for studying a dynamic change of heavy metals. The input pathways of heavy metals to farmland soil mainly include atmospheric dust fall, irrigation water, and a pesticide and a fertilizer, and the output pathways mainly include surface runoff, infiltration, and output through a crop. A change in heavy metal accumulation is discussed by calculating an equilibrium relationship between input and output fluxes. However, this method ignores the contribution of a natural source (the soil parent material) to the heavy metal content in soil, and lacks more detailed source apportionment, such as quantitative contributions of a transportation source and an industrial source.

### SUMMARY

An objective of the present application is to provide an effective source identification and flux prediction method for farmland soil polluted by heavy metals, providing a more accurate source apportionment result and reasonable prediction for heavy metal pollution of the farmland soil.

A study example of the present application is heavy metals in farmland soil of a region affected by a nonferrous metal dressing and smelting site.

To achieve the above technical objective and achieve the foregoing technical effects, the present application is achieved through the following technical solutions:

The present application provides a method for predicting heavy metal accumulation in soil based on an emission inventory and a receptor model, including the following steps:
S1: calculating a spatial-temporal change of a heavy metal accumulation flux in surface soil, and establishing a heavy metal pollutant input-output flux inventory with atmospheric dust fall, irrigation water, and a pesticide and a fertilizer as heavy metal input flux sources to farmland soil and surface runoff water and a crop as output fluxes to investigate a dynamic equilibrium relationship of heavy metal accumulation in soil, where a depth of the surface soil is 0-20 cm;
S2: attributing heavy metal sources of farmland soil of a region affected by a nonferrous metal dressing and smelting site to the atmospheric dust fall, the irrigation water, the pesticide, and the fertilizer;
where heavy metal output pathways of the farmland soil are the surface runoff and the crop; and
a heavy metal input flux and a heavy metal output flux are annual heavy metal input and output masses per unit area in the farmland soil of the region affected by the nonferrous metal dressing and smelting site, respectively, in units of g/y·ha; and
calculating the heavy metal input flux and the heavy metal output flux of a monitored region;
S3: carding an input flux and output flux inventory according to calculation results of step S2, and calculating a heavy metal input-output equilibrium per unit area per year in surface (0-20 cm) farmland soil of the region affected by the nonferrous metal dressing and smelting site by establishing an inventory, with a calculation formula being as shown in formula (1):

Δsoil=Σinputs−Σoutputs formula (1)
where in formula (1), Asoil represents an annual heavy metal variation of soil per unit area; and
Σinputs and Σoutputs represent the heavy metal input flux and the heavy metal output flux of soil per unit area calculated in step S2, respectively, in units of g/y·ha;
S4: according to the mass equilibrium formula (1) in step S3, calculating a heavy metal accumulation flux, and calculating an annual heavy metal accumulation speed per unit area in the farmland soil of the region affected by the nonferrous metal dressing and smelting site by formula (2):

$$DVsoil = \frac{{Δ}_{soil}}{h * ρ soil} * {10}^{3} ; \qquad formula ( 2 )$$

where in formula (2):
DVₛₒᵢₗ represents a heavy metal accumulation speed in soil per unit mass, in units of mg/y·kg;
Δₛₒᵢₗ represents an annual heavy metal accumulation flux in soil per unit area, in units of g/y·ha;
h represents a soil depth of the monitored region, which is 1 m;
ρₛₒᵢₗ represents an average soil density of the monitored region; and
the ρₛₒᵢₗ is 1540 kg/m³; and

S5: with a heavy metal content in the surface soil in step S1 as a study object, employing a positive matrix factorization (PMF) model to perform data analysis, extracting a plurality of factors, identifying the plurality of factors as different source categories with marking components, and then calculating specific contributions of different factors to the heavy metal content in the soil by multiple linear regression, with a specific formula being as shown in formula (8):

$${x}_{ij} = {\sum }_{k = 1}^{p} {g}_{ik} {f}_{kj} + {e}_{ij} ; \qquad formula ( 8 )$$

where in formula (8), xᵢⱼ represents a concentration of a jth heavy metal in an ith sample, in units of mg/kg;
p represents a number of factors affecting a heavy metal concentration in a sample;
gᵢₖ represents a mass concentration of a kth factor to the ith sample;
fₖⱼ represents a mass concentration of a jth element in the ith sample; and
eᵢⱼ represents a residual of the jth element in the ith sample; and
minimizing an objective function Q by using a weighted least square method, as shown in formula (9), to obtain a model result:

$$Q = {\sum }_{i = 1}^{n} {{\sum }_{j = 1}^{m} [ \frac{{x}_{ij} - {\sum }_{k = 1}^{p} {g}_{ik} {f}_{kj}}{{u}_{ij}} ]}^{2} ; \qquad formula ( 9 )$$

where in formula (9), uᵢⱼ represents an uncertainty value of the jth heavy metal in the ith sample, which is related to laboratory conditions and a test method, and quantized by formula (10):

$${u}_{ij} = \{ \begin{matrix}\frac{5}{6} \times MDL \\ \sqrt{{( δ \times {x}_{ij} )}^{2} + {( 0.5 \times MDL )}^{2}}\end{matrix} Xij \leq MDL , Xij > MDL ; \qquad ( 10 )$$

where in formula (10), 8 represents a relationship standard deviation of a heavy metal concentration; and MDL represents a method detection limit.

Further, step S1 may specifically include the following contents:

### (1) Soil

Sampling rule: using a five-point sampling method, where the five-point sampling method refers to collecting samples at four vertexes and a center of a square and then mixing the samples; a sampling depth is 0-20 cm (surface soil); in an entire monitored region, a distribution of sampling points conforms to the following principle: starting from a nonferrous metal dressing and smelting slag yard, a sampling point is determined in units of 200 meter in a migration path, sampling points being as shown in FIG. 1;
the sample treatment method of the soil includes: air-drying soil samples, and then sieving all the soil samples using a 2 mm nylon screen, where a filter mesh is stored in a polyethylene bottle; a part of each sample is further ground to pass through a 0.15 mm nylon screen; and after digestion, a total heavy metal content is analyzed; and
the digestion includes the following specific steps: putting 0.1 g of sample into a digestion pot, adding 6 mL of nitric acid, using an acid evaporator for pretreatment at 120° C. for 30 min, cooling, supplementing 1 mL of nitric acid, 2 mL of hydrofluoric acid, and 3 mL of hydrochloric acid, and in a microwave instrument, setting a temperature time gradient as follows: maintaining the temperature at 150° C. for 10 min, at 180° C. for 5 min, and at 200° C. for 25 min; after the completion of microwaving, evaporating acid in the acid evaporator at 170° C. for 30 min, during which 1 mL of perchloric acid is added; evaporating acid to about 2 mL, cooling and diluting to 25 mL, and then testing a heavy metal (lead, zinc, arsenic, copper, or cadmium) concentration using an inductively coupled plasma-mass spectrometer (ICP-MS).

### (2) Surface Water

Sampling rule: the surface water is divided into river water, irrigation water, and surface runoff water;
a sampling method for the river water includes: setting sampling points upstream, midstream, and downstream of a river, and collecting river water samples in polyethylene bottles, with a sampling quantity being 500 mL at a time;
a sampling method for the irrigation water includes: collecting an irrigation water sample in a polyethylene bottle when a farmer performs irrigation, with a sampling quantity being 500 mL at a time;
a sampling method for the surface runoff water includes: for runoff from a farmland water outlet to a river, collecting a surface runoff water sample in a polyethylene bottle at the farmland water outlet, while detecting and recording a flow rate;
the sample treatment method of the surface water includes: for each water sample, sealing and shaking well the water sample, and bringing the water sample back to a laboratory for digestion; and
the digestion includes the following specific steps: putting a water sample into a crucible, adding 6 mL of nitric acid and 2 mL of hydrogen peroxide, putting a cover on the crucible, placing the crucible on an electric heating plate for heating at 120° C. for two hours, removing the cover, continuously heating until about 5 mL of solution remains, diluting, and then (MS) testing a heavy metal (lead, zinc, arsenic, copper, or cadmium) concentration using the inductively coupled plasma-mass spectrometer (ICP-MS).

### (3) Atmospheric Dust Fall

A sampling method for the atmospheric dust fall includes: starting from a nonferrous metal dressing and smelting slag yard, setting atmospheric dust fall collection points in a migration path, placing a dust collection jar at a position 5 meter above the ground to avoid a suspended matter in soil from affecting the collection of the atmospheric dust fall, and adding 5 mL of glycol to each dust collection jar to avoid a bacteria breeding exogenous material from affecting a sample composition; then adding 2% of HNO₃ to prevent types of elements from changing, where dry and wet deposition occurs without separation when collecting a sample; and packaging the sample in a polyethylene bottle and delivering the polyethylene bottle to the laboratory as soon as possible for treatment;
the sample treatment method of the atmospheric dust fall includes: standing an atmospheric dust fall sample for 2-3 days, centrifuging in a centrifuge at 3500 rpm for 10 min, transferring a supernatant liquid to a glass bottle, and measuring a volume thereof; transferring remaining precipitate to a beaker, drying under a condition of 60° C. to a constant weight, and recording a mass thereof, where the supernatant liquid is a wet deposition sample; the precipitate is a dry deposition sample; digesting the treated dry and wet deposition samples and then detecting a total heavy metal content;
a digestion method of the wet deposition sample is the same as the digestion method of the surface water; and the digestion of the dry deposition sample includes the following specific steps: putting 0.1 g of sample into a digestion pot, adding 5 mL of hydrochloric acid, 5 mL of hydrofluoric acid, and 1 mL of hydrogen peroxide, and in a microwave instrument, setting a temperature time gradient as follows: maintaining the temperature at 150° C. for 10 min, at 180° C. for 5 min, and at 200° C. for 25 min; after the completion of microwaving, evaporating acid in the acid evaporator at 170° C. for 30 min, during which 1 mL of perchloric acid is added; evaporating acid to about 2 mL, cooling and diluting to 25 mL, and then testing a heavy metal (lead, zinc, arsenic, copper, or cadmium) concentration using the inductively coupled plasma-mass spectrometer (ICP-MS).

### (4) Crop

A sampling method for the crop includes: collecting a whole plant of the corn crop at harvest time, uniformly distributing sampling points in a monitored region, removing soil from a root as much as possible, then splitting the corn plant into three parts: root, stem leaf, and fruit, putting the three parts in sample bags separately, and bringing the sample bags back to the laboratory as soon as possible for treatment;
the sample treatment method of the crop includes: using tap water to wash off soil and impurities from a collected corn sample (including the root, the stem, the leaf, and the fruit) first, then repeatedly washing with deionized water, drying in a drying oven under a condition of 105° C. for 2 h, then completely drying at 60° C. poor for 48 h, and recording a crop mass after drying; grinding the dried sample, and then testing a total heavy metal content after digestion; and
the digestion includes the following specific steps: putting about 0.1 g of sample into a digestion pot, adding 8 mL of nitric acid for soaking overnight, then adding 1 mL of hydrogen peroxide, microwaving after reacting for a while, and setting a temperature time gradient as follows: maintaining the temperature at 150° C. for 10 min and at 190° C. for 25 min, where a power is determined by a number of pots; after the completion of microwaving, putting the sample in an acid evaporator for acid evaporation at 170° C. for 30 min until the remaining sample is about 2 mL, cooling and diluting, and then testing a heavy metal (lead, zinc, arsenic, copper, or cadmium) concentration using the inductively coupled plasma-mass spectrometer (ICP-MS).
   (5) Fertilizer and pesticide

According to farming habits of local farmers, at different growth stages of corn, 6 fertilizer and pesticide samples are obtained locally; the fertilizer samples are stored in sealable bags and the pesticide samples are stored in clean polyethylene bottles, and brought back to the laboratory for treatment;
the pesticide samples include a Spodoptera litura multicapsid nucleopolyhedrovirus sample, a thiamethoxam-lambda-cyhalothrin sample, a nicosulfuron sample, and a nicosulfuron-dicamba-fluroxypyr sample, and the fertilizer samples includes a urea sample and a compound fertilizer sample;
the sample treatment method of the fertilizer and the pesticide includes: testing a total heavy metal content after sample digestion; and the digestion includes the following specific steps: putting 5 mL of sample into a 100 mL beaker, adding 50% of nitric acid for digestion to be almost dry, dissolving with a 5% nitric acid solution and diluting to 50 mL, and then testing a heavy metal (lead, zinc, arsenic, copper, or cadmium) concentration using the inductively coupled plasma-mass spectrometer (ICP-MS).

Further, step S2 may specifically include: accounting specifically according to formulas (3) to (7):

### (1) An Atmospheric Dust Fall Input Flux

$${I}_{A t} = ( {C}_{w} {V}_{w} + {C}_{d} {W}_{d} ) \times 100 / S ; \qquad formula ( 3 )$$

where in formula (3):
I_{At} represents a heavy metal flux into the farmland soil of the monitored region through the atmospheric dust fall, in units of g/ha·y;
C_{w} and C_{d} represent heavy metal concentrations in wet deposition and dry deposition, in units of ug/L and ug/g, respectively;
V_{w} and W_{d} represent quantities of wet deposition and dry deposition, in units of L and g, respectively;
S represents an area of a sampling bottle opening, in units of cm²; and 100 is a unit conversion coefficient;

### (2) A Pesticide and Fertilizer Input Flux

$${I}_{Fer} = {\sum }_{j = 1}^{n} {F}_{i , j} {C}_{i , j} {10}^{- 6} ; \qquad formula ( 4 )$$

where in formula (4), I_{Fer} represents a heavy metal pesticide and fertilizer input flux, in units of g/ha y;
Fᵢⱼ represents an actual application rate of the pesticide and the fertilizer, in units of g/y·ha;
Cᵢⱼ represents a content of an element in the fertilizer and the pesticide, in units of mg/kg or ug/mL; and n represents a number of types of fertilizers applied at a sampling point;

### (3) An Irrigation Water Input Flux

$${I}_{I r} = V {C}_{i} \cdot {10}^{- 6} ; \qquad formula ( 5 )$$

where in formula (5):
I_{Ir} represents an input quantity of a heavy metal (i) from the irrigation water, in units of g/y·ha;
V represents an irrigation water application quantity, in units of L/y·ha; and
Cᵢ represents a concentration of an element (i) in the irrigation water, in units of ug/L;

### (4) A Surface Runoff Output Flux

$${O}_{Run} = {C}_{i} VR \times \frac{100}{S} ; \qquad formula ( 6 )$$

where in formula (6):
O_{Run} represents an output quantity of a heavy metal (i) in the runoff, in units of g/y·ha;
Cᵢ represents a concentration of the heavy metal (i) in the runoff, in units of ug/L;
V represents a volume of the surface runoff water, in units of L;
R represents a ratio of annual average precipitation and rainfall during study, which is unitless; and
S represents an area of a sampling bottle neck, in units of cm²; and

### (5) A Crop Output Flux

$${O}_{Crop} = {\sum }_{j = 1}^{n} {N}_{e , j} {C}_{i , e , j} + {\sum }_{j = 1}^{n} {N}_{n , j} {C}_{i , n , j} + {\sum }_{j = 1}^{n} {N}_{p , j} {C}_{i , n , j} ; \qquad formula ( 7 )$$

where in formula (7):
O_{Crop} represents an output of a heavy metal (i) in the crop (j), in units of g/y·ha;
N_{e,j} represents a quantity of the root part of the annual crop (j);
C_{i,e,j} represents a concentration of the heavy metal (i) in the root part of the crop (j) in units of ug/g;
N_{n,j} represents a content of a stem leaf part (j) of the annual crop, in units of g/y·ha;
C_{i,n,j} represents a concentration of an element (i) in the stem leaf part (j) of the crop, in units of ug/g;
N_{p,j} represents a number of fruits (p) per year, in units of g/y·ha;
C_{i,p,j} represents a concentration of a heavy metal i in the fruits (p) of the crop, in units of ug/g; and
n represents a number of harvested parts.

The present application has the following beneficial effects:

An emission inventory is established to reveal a dynamic equilibrium relationship of heavy metal accumulation in soil: by establishing the heavy metal pollutant input-output flux inventory, input and output fluxes of heavy metals in farmland soil can be quantitatively calculated in the present application, which is conducive to revealing the dynamic equilibrium relationship of heavy metal accumulation in soil and predicting an accumulation change of the heavy metals in the farmland soil. The inventory of input sources and output pathways main includes: atmospheric dust fall, irrigation water, and a pesticide and a fertilizer as heavy metal input flux sources to the farmland soil and surface runoff water and a crop as output fluxes. By calculating the equilibrium relationship of the input and output fluxes of the heavy metals, the sources and destinations of the heavy metals are quantitatively calculated.

The present application attempts to introduce a PMF model to deeply discuss the contribution sources of the heavy metal pollution in the farmland soil. The result of the emission inventory shows that the heavy metals mainly come from the atmospheric dust fall. This is an intuitive representation of test data. However, the PMF model may be employed to conduct a discussion on the internal mathematical logic of a heavy metal content in soil. In fact, this method also assists in explaining the sources of heavy metals from the atmospheric dust fall in a monitored region and realizes detailed source apportionment of heavy metals in the region.

Therefore, the present application combines the receptor model (the PMF model) with the emission inventory. On the basis of investigating the heavy metal accumulation change, the source of heavy metal accumulation is further apportioned, and contribution degrees of a transportation source, a natural source, an industrial source, and an agricultural source are quantitatively calculated. The source apportionment and heavy metal accumulation prediction are combined. The technical solution may provide theoretical guidance for the prevention and accurate control of the heavy metal pollution in soil. Based on these data, effective pollution treatment measure and management strategy may be formulated to reduce the heavy metal accumulation in soil and reduce the risks to the farmland ecosystem and the human health.

Certainly, the implementation of any product in the present application does not necessarily need to achieve all of the above advantages.

### BRIEF DESCRIPTION OF THE DRAWINGS

To describe the technical solutions in the embodiments of the present application more clearly, the drawings required for describing the embodiments are briefly described below. Apparently, the drawings in the following description show merely some embodiments of the present application, and those of ordinary skill in the art may still derive other drawings from these drawings without creative efforts.

FIG. 1 shows a field sample collection image;

FIG. 2 shows an image of a distribution of atmospheric dust fall collection points;

FIG. 3 shows an image of a distribution of pesticide and fertilizer collection points;

FIG. 4 shows an image of a distribution of irrigation water collection points;

FIG. 5 shows an image of a distribution of surface runoff collection points; and

FIG. 6 shows an image of a distribution of crop collection points.

### DETAILED DESCRIPTION OF THE EMBODIMENTS

The technical solutions in the embodiments of the present application will be described clearly and completely below with reference to the accompanying drawings in the embodiments of the present application. Apparently, the described embodiments are merely some rather than all of the embodiments of the present application. All other embodiments derived from the embodiments of the present application by those of ordinary skill in the art without creative efforts shall fall within the protection scope of the present application.

### Example 1

This example provides a method for predicting heavy metal accumulation in soil based on an emission inventory and a receptor model, including the following steps.

In step S1, a spatial-temporal change of a heavy metal accumulation flux in surface soil (0-20 cm), and a heavy metal pollutant input-output flux inventory is established with atmospheric dust fall, irrigation water, and a pesticide and a fertilizer as heavy metal input flux sources to farmland soil and surface runoff water and a crop as output fluxes to investigate a dynamic equilibrium relationship of heavy metal accumulation in soil.

In step S2: heavy metal sources of farmland soil of a region affected by a nonferrous dressing and smelting site are attributed to the atmospheric dust fall, the irrigation water, the pesticide, and the fertilizer;
heavy metal output pathways of the farmland soil are the surface runoff and the crop;
and a heavy metal input flux and a heavy metal output flux are annual heavy metal input and output masses per unit area in the region affected by the nonferrous metal dressing and smelting site, respectively, in units of g/y·ha; and
three heavy metal input fluxes of the atmospheric dust fall, the irrigation water, the pesticide, and the fertilizer and the heavy metal output fluxes of the surface runoff and the crop in a monitored region are calculated.

In step S3: an input flux and output flux inventory is carded according to calculation results of step S2, and a heavy metal input-output equilibrium per unit area per year in farmland soil by establishing an inventory, with a calculation formula being as shown in formula (1):

Δsoil=Σinputs−Σoutputs formula (1); and
where in formula (1), Δsoil represents an annual heavy metal variation of soil per unit area; and
Σinputs and Σoutputs represent the heavy metal input flux and the heavy metal output flux of soil per unit area calculated in step S2, respectively, in units of g/y·ha.

In step S4: according to the mass equilibrium formula (1) in step S3, a heavy metal accumulation flux is calculated, and an annual heavy metal accumulation speed per unit mass of farmland soil by the following formula:

$$DVsoil = \frac{{Δ}_{soil}}{h * ρ soil} * {10}^{3} ; \qquad formula ( 2 )$$

where:
in formula (2), DVₛₒᵢₗ represents a heavy metal accumulation speed in soil per unit mass, in units of mg/y kg;
Δₛₒᵢₗ represents an annual heavy metal accumulation flux in soil per unit area, in units of g/y·ha;
h represents a soil depth of the monitored region, which is 1 m; and
ρsoil represents an average soil density of the monitored region, which is 1540 kg/m³.

In step S5, with a heavy metal content in the surface soil in step S1 as a study object, a positive matrix factorization (PMF) model is employed to perform data analysis; a plurality of factors are extracted; the plurality of factors are identified as different source categories with marking components, and then specific contributions of different factors to the heavy metal content in the soil are calculated by multiple linear regression, with a specific formula being as shown in formula (8):

$${x}_{ij} = {\sum }_{k = 1}^{p} {g}_{ik} {f}_{kj} + {e}_{ij} ; \qquad formula ( 8 )$$

where in formula (8), xᵢⱼ represents a concentration of a jth heavy metal in an ith sample, in units of mg/kg;
p represents a number of factors affecting a heavy metal concentration in a sample;
gᵢₖ represents a mass concentration of a kth factor to the ith sample;
fᵢⱼ represents a mass concentration of a jth element in the ith sample; and
eᵢⱼ represents a residual of the jth element in the ith sample; and
minimizing an objective function Q by using a weighted least square method, as shown in formula (9), to obtain a model result:

$$Q = {\sum }_{i = 1}^{n} {{\sum }_{j = 1}^{m} [ \frac{{x}_{ij} - {\sum }_{k = 1}^{p} {g}_{ik} {f}_{kj}}{{u}_{ij}} ]}^{2} ; \qquad formula ( 9 )$$

where in formula (9), uᵢⱼ represents an uncertainty value of the jth heavy metal in the ith sample, which is related to laboratory conditions and a test method, and quantized by formula (10):

$${u}_{ij} = \{ \begin{matrix}\frac{5}{6} \times MDL \\ \sqrt{{( δ \times {x}_{ij} )}^{2} + {( 0.5 \times MDL )}^{2}}\end{matrix} Xij \leq MDL , Xij > MDL . \qquad ( 10 )$$

where in formula (10), & represents a relationship standard deviation of a heavy metal concentration; and MDL represents a method detection limit.

In this example, step S1 specifically includes the following steps.

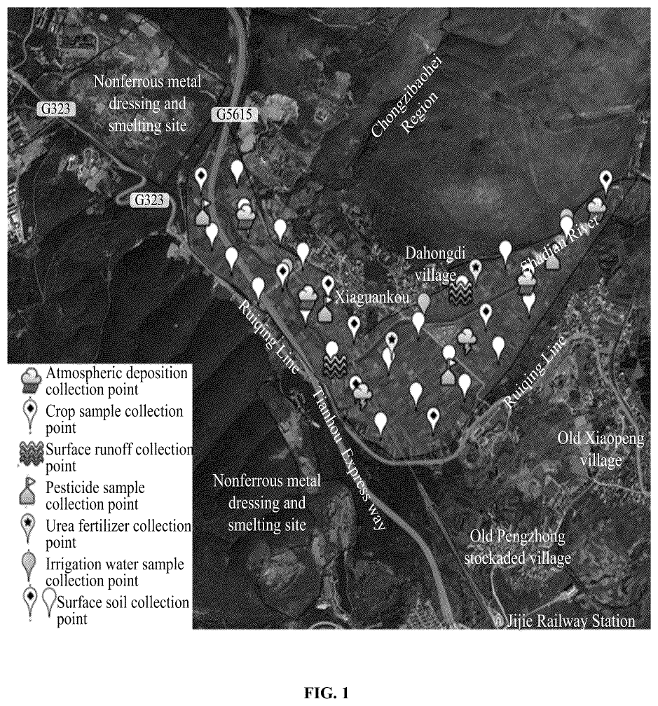

(1) Soil sampling rule: a five-point sampling method is used, where the five-point sampling method refers to collecting samples at four vertexes and a center of a square and then mixing the samples; a sampling depth is 0-20 cm; in an entire monitored region, a distribution of sampling points conforms to the following principle: starting from a nonferrous metal dressing and smelting slag yard, a sampling point is determined in units of 200 meter in a migration path, sampling points being as shown in FIG. 1.

Sample treatment method: samples are air-dried; soil samples are air-dried, and then all the air-dried soil samples are sieved using a 2 mm nylon screen, and a filter mesh is stored in a polyethylene bottle; a part of each sample is further ground to pass through a 0.15 mm nylon screen; and after digestion, a total heavy metal content is analyzed.

The digestion includes the following specific steps: 0.1 g of soil sample is put into a digestion pot; 6 mL of nitric acid is added; an acid evaporator is used for pretreatment at 120° C. for 30 min; cooling is performed; 1 mL of nitric acid, 2 mL of hydrofluoric acid, and 3 mL of hydrochloric acid are supplemented; and in a microwave instrument, a temperature time gradient is set as follows: the temperature is maintained at 150° C. for 10 min, at 180° C. for 5 min, and at 200° C. for 25 min. After the completion of microwaving, acid is evaporated in the acid evaporator at 170° C. for 30 min, during which 1 mL of perchloric acid is added. The acid is evaporated to about 2 mL, followed by cooling and diluting to 25 mL, and then a heavy metal (lead, zinc, arsenic, copper, or cadmium) concentration is tested using an inductively coupled plasma-mass spectrometer (ICP-MS).

### (2) Surface Water

Sampling rule: the surface water is divided into river water, irrigation water, and surface runoff water.

For the river water, sampling points are set upstream, midstream, and downstream of a river, and river water samples are collected in polyethylene bottles, with a sampling quantity being 500 mL at a time.

For the irrigation water, an irrigation water sample is collected in a polyethylene bottle when a farmer performs irrigation, with a sampling quantity being 500 mL at a time.

For the surface runoff water, for runoff from a farmland water outlet to a river, a surface runoff water sample is collected in a polyethylene bottle at for the farmland water outlet, while a flow rate is detected and recorded.

Sample treatment method: all the water samples are sealed and then shaken well, and brought back to a laboratory for digestion. The digestion includes the following specific steps: a sample is put into a crucible; 6 mL of nitric acid and 2 mL of hydrogen peroxide are added; a cover is put on the crucible; the crucible is placed on an electric heating plate for heating at 120° C. for two hours; the cover is removed; heating is continued until about 5 mL of solution remains, followed by diluting, and then a heavy metal (lead, zinc, arsenic, copper, or cadmium) concentration is tested using the inductively coupled plasma-mass spectrometer (ICP-MS).

### (3) Atmospheric Dust Fall

Sampling method: starting from a nonferrous metal dressing and smelting slag yard, atmospheric dust fall collection points are set in a migration path; a dust collection jar is placed at a position 5 meter above the ground to avoid a suspended matter in soil from affecting the collection of the atmospheric dust fall, and 5 mL of glycol is added to each dust collection jar to avoid a bacteria breeding exogenous material from affecting a sample composition; then 2% of HNO₃ is added to prevent types of elements from changing; dry and wet deposition occurs without separation when collecting a sample; and the sample is packaged in a polyethylene bottle and delivered to the laboratory as soon as possible for treatment.

Sample treatment method: an atmospheric dust fall sample is allowed to stand for 2-3 days, and then centrifuged in a centrifuge at 3500 rpm for 10 min; a supernatant liquid is transferred to a glass bottle, and a volume thereof is measured; remaining precipitate is transferred to a beaker, and dried under a condition of 60° C. to a constant weight, and a mass thereof is recorded; the supernatant liquid is a wet deposition sample; and after the treated dry and wet deposition samples are digested, a total heavy metal content is detected.

A digestion method of the wet deposition sample is the same as the digestion method of the surface water; and the digestion of the dry deposition sample includes the following specific steps: 0.1 g of sample is put into a digestion pot; 5 mL of hydrochloric acid, 5 mL of hydrofluoric acid, and 1 mL of hydrogen peroxide are added; and in a microwave instrument, a temperature time gradient is set as follows: the temperature is maintained at 150° C. for 10 min, at 180° C. for 5 min, and at 200° C. for 25 min; after the completion of microwaving, acid is evaporated in the acid evaporator at 170° C. for 30 min, during which 1 mL of perchloric acid is added; acid is evaporated to about 2 mL, followed by cooling and diluting to 25 mL, and then a heavy metal (lead, zinc, arsenic, copper, or cadmium) concentration is tested using the inductively coupled plasma-mass spectrometer (ICP-MS).

### (4) Crop

Sampling method: a whole plant of the corn crop is collected at harvest time; sampling points are uniformly distributed in a monitored region; soil is removed from a root as much as possible, and then the corn plant is split into three parts: root, stem leaf, and fruit; the three parts are put in sample bags separately, and the sample bags are brought back to the laboratory as soon as possible for treatment.

Sample treatment method: a collected corn sample (including the root, the stem, the leaf, and the fruit) is washed with tap water first to remove soil and impurities, then repeated washed with deionized water, dried in a drying oven under a condition of 105° C. for 2 h, and then completely dried at 60° C. poor for 48 h, and a crop mass after drying is recorded; the dried sample is ground, and then a total heavy metal content is tested after digestion.

The digestion includes the following specific steps: about 0.1 g of sample is put into a digestion pot; 8 mL of nitric acid is added for soaking overnight, and then 1 mL of hydrogen peroxide is added; microwaving is performed after reacting for a while, and a temperature time gradient is set as follows: the temperature is maintained at 150° C. for 10 min and at 190° C. for 25 min, where a power is determined by a number of pots; after the completion of microwaving, the sample is put in an acid evaporator for acid evaporation at 170° C. for 30 min until the remaining sample is about 2 mL, followed by cooling and diluting, and then a heavy metal (lead, zinc, arsenic, copper, or cadmium) concentration is tested using the inductively coupled plasma-mass spectrometer (ICP-MS).

### (5) Fertilizer and Pesticide

According to farming habits of local farmers, at different growth stages of corn, 6 fertilizer and pesticide samples are obtained locally; the fertilizer samples are stored in sealable bags and the pesticide samples are stored in clean polyethylene bottles, and brought back to the laboratory for treatment.

Sample treatment method: the pesticide samples include a Spodoptera litura multicapsid nucleopolyhedrovirus sample, a thiamethoxam-lambda-cyhalothrin sample, a nicosulfuron sample, and a nicosulfuron-dicamba-fluroxypyr sample, and the fertilizer samples include a urea sample and a compound fertilizer sample.

A total heavy metal content is tested after sample digestion. The digestion includes the following specific steps: 5 mL of sample is put into a 100 mL beaker; 50% of nitric acid is added for digestion to be almost dry; a 5% nitric acid solution is used for dissolving and diluting to 50 mL, and then a heavy metal (lead, zinc, arsenic, copper, or cadmium) concentration is tested using the inductively coupled plasma-mass spectrometer (ICP-MS).

In this example, step S2 specifically includes the following step: accounting is specifically performed according to formulas (3) to (7):

### (1) An Atmospheric Dust Fall Input Flux

$${I}_{At} = ( {C}_{w} {V}_{w} + {C}_{d} {W}_{d} ) \times 100 / S ; \qquad formula ( 3 )$$

where in formula (3), I_{At} represents a heavy metal flux into the farmland soil of the monitored region through the atmospheric dust fall, in units of g/ha y;
C_{w} and C_{d} represent heavy metal concentrations in wet deposition and dry deposition, in units of ug/L and ug/g, respectively;
V_{w} and W_{d} represent quantities of wet deposition and dry deposition, in units of L and g, respectively;
S represents an area of a sampling bottle opening, in units of cm²; and 100 is a unit conversion coefficient;

### (2) A Pesticide and Fertilizer Input Flux

$${I}_{Fer} = {\sum }_{j = 1}^{n} {F}_{i , j} {C}_{i , j} {10}^{- 6} ; \qquad formula ( 4 )$$

where in formula (4), I_{Fer} represents a heavy metal pesticide and fertilizer input flux, in units of g/ha y;
Fᵢⱼ represents an actual application rate of the pesticide and the fertilizer, in units of g/y·ha;
Cᵢⱼ represents a content of an element in the fertilizer and the pesticide, in units of mg/kg or ug/mL; and
n represents a number of types of fertilizers applied at a sampling point;

### (3) An Irrigation Water Input Flux

$${I}_{Ir} = {VC}_{i} \cdot {10}^{- 6} ; \qquad formula ( 5 )$$

where in formula (5):
I_{Ir} represents an input quantity of a heavy metal (i) from the irrigation water, in units of g/y·ha;
V represents an irrigation water application quantity, in units of L/y·ha; and
Cᵢ represents a concentration of an element (i) in the irrigation water, in units of ug/L;

### (4) A Surface Runoff Output Flux

$${O}_{Run} = {C}_{i} VR \times \frac{100}{S} ; \qquad formula ( 6 )$$

where in formula (6):
O_{Run} represents an output quantity of a heavy metal (i) in the runoff, in units of g/y·ha;
Cᵢ represents a concentration of the heavy metal (i) in the runoff, in units of ug/L;
V represents a volume of the surface runoff water, in units of L;
R represents a ratio of annual average precipitation and rainfall during study, which is unitless; and
S represents an area of a sampling bottle neck, in units of cm²; and

### (5) A Crop Output Flux

$${O}_{Crop} = {\sum }_{j = 1}^{n} {N}_{e , j} {C}_{i , e , j} + {\sum }_{j = 1}^{n} {N}_{n , j} {C}_{i , n , j} + {\sum }_{j = 1}^{n} {N}_{p , j} {C}_{i , n , j} ; \qquad formula ( 7 )$$

where in formula (7):
O_{Crop} represents an output of a heavy metal (i) in the crop (j), in units of g/y·ha;
Nₑⱼ represents a quantity of the root part of the annual crop (j);
Cᵢₑⱼ represents a concentration of the heavy metal (i) in the root part of the crop (j) in units of ug/g;
Nₙⱼ represents a content of a stem leaf part (j) of the annual crop, in units of g/y·ha;
C_{i,n,j} represents a concentration of an element (i) in the stem leaf part (j) of the crop, in units of ug/g;
Nₚⱼ represents a number of fruits (p) per year, in units of g/y·ha;
C_{i,p,j} represents a concentration of a heavy metal i in the fruits (p) of the crop, in units of ug/g; and
n represents a number of harvested parts.

### Example 2

### Sample Collection:

Taking a lead-zinc smelting industrial zone in Gejiu, Yunnan in southwest China as an example, field investigation and sampling are conducted on a farmland region in the zone. All sampling points are as shown in FIG. 1.

Calculation of input and output fluxes in the affected region:

After experimental analysis of collected samples, the corresponding calculation formulas in step S2 are adopted for calculation.

### I. Calculation of Input Fluxes

### (1) An Atmospheric Dust Fall Input Flux

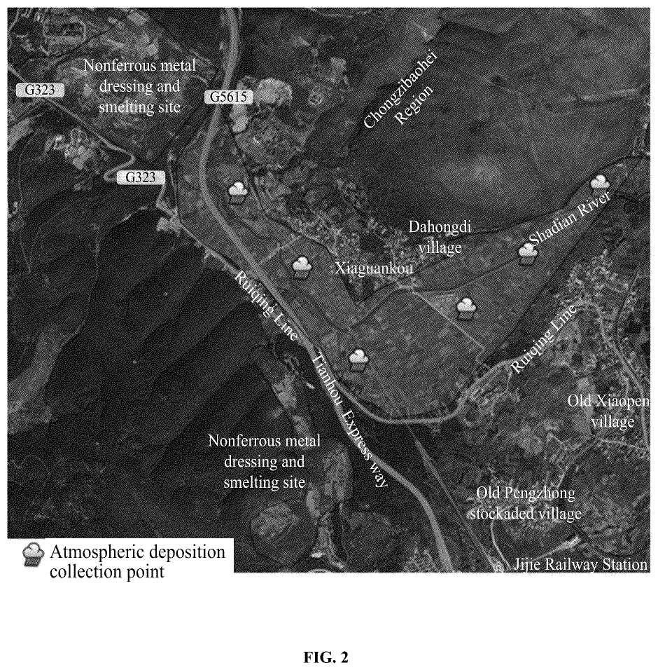

In the monitored region, 4 dust collection jars are arranged within the range of 2 kilometers, as shown in FIG. 2.

TABLE 2-1
Atmospheric Dust Fall Input Flux

| Input Flux (g/y · ha) | Pb | As | Cu | Cd | Zn |
|---|---|---|---|---|---|
| Atmospheric dust fall | 226.43 | 262.74 | 77.51 | 28.06 | 802.00 |

The settlement amount of the farmland per hectare in the monitored region is 38.253 kg. According to the calculation method of the heavy metal input flux from the atmospheric dust fall, the input fluxes of Pb, As, Cu, Cd, and Zn in the monitored region are 226.43 g/y·ha, 262.74 g/y·ha, 77.51 g/y·ha, 28.06 g/y·ha, and 802.00 g/y·ha, respectively.

### (2) A Pesticide and Fertilizer Input Flux

TABLE 2-2
Pesticide and Fertilizer Input Flux

| Input Flux (g/y · ha) | Pb | As | Cu | Cd | Zn |
|---|---|---|---|---|---|
| Fertilizer and | 0.07 | 0.04 | 0.71 | 0.00 | 0.29 |
| Pesticide | | | | | |

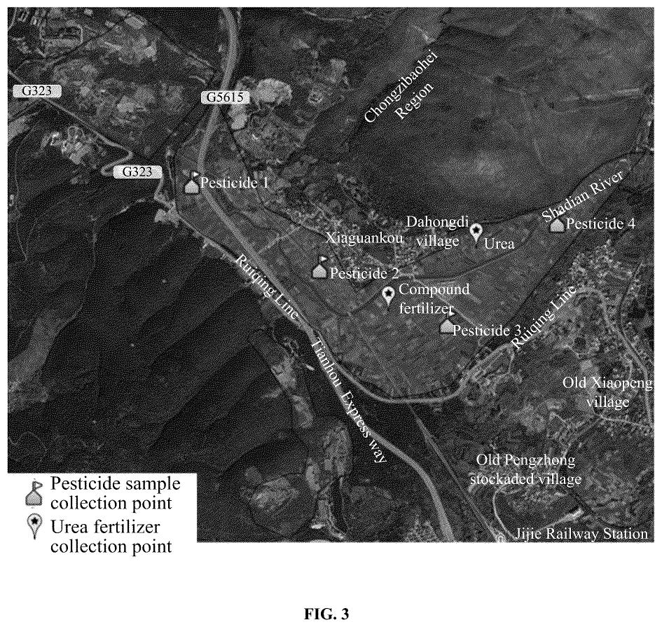

Four pesticide samples of Spodoptera litura multicapsid nucleopolyhedrovirus, thiamethoxam-lambda-cyhalothrin, nicosulfuron, and nicosulfuron-dicamba-fluroxypyr, and two fertilizer samples of urea and compound fertilizer are collected in the farmland in the monitored region. The pesticide and fertilizer sampling points are as shown in FIG. 3, and the usage amounts of pesticides and fertilizers per year are investigated from local farmers. The pesticide and fertilizer heavy metal input fluxes are calculated according to formula (2). The input fluxes of Pb, As, Cu, Cd, and Zn in the farmland soil of the monitored region are shown as 0.07 g/y·ha, 0.04 g/y·ha, 0.71 g/y·ha, 0.00 g/y·ha, and 0.29 g/y·ha, respectively.

### (3) An Irrigation Water Input Flux

TABLE 2-3
Irrigation Water Input Flux

| Input Flux (g/y · ha) | Pb | As | Cu | Cd | Zn |
|---|---|---|---|---|---|
| Irrigation water | 0.26 | 2.72 | 0.12 | 0.01 | 0.88 |

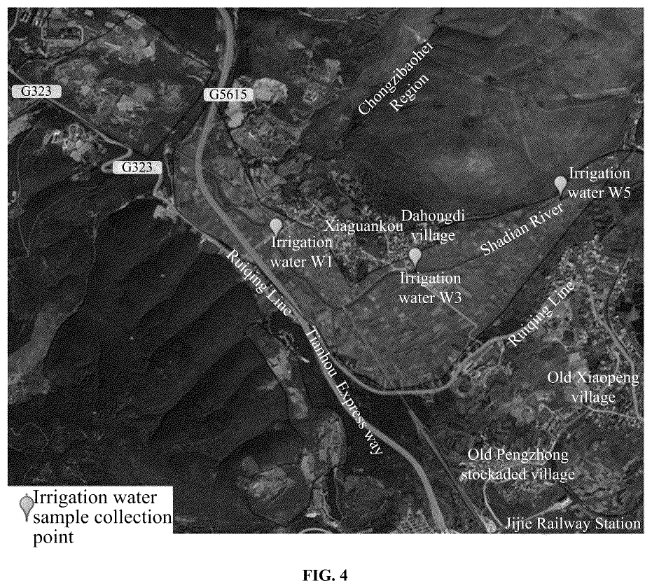

In 2023, the volume of irrigation water for the farmland soil per mu in the monitored region is about 16250 L. according to the measurement of the average metal concentration of the collected irrigation water samples, the sampling points are as shown in FIG. 4, and an annual usage amount of the irrigation water is investigated from the local farmers. According to formula (3), the input fluxes of Pb, As, Cu, Cd, and Zn in the farmland soil of the monitored region are calculated as 0.26 g/y·ha, 2.72 g/y·ha, 0.12 g/y·ha, 0.01 g/y·ha, and 0.88 g/y·ha, respectively.

### II. Calculation of Output Fluxes

### (1) A Surface Runoff Output Flux

TABLE 2-4
Surface Runoff Output Flux

| Input Flux (g/y · ha) | Pb | As | Cu | Cd | Zn |
|---|---|---|---|---|---|
| Surface runoff | 0.07 | 0.13 | 0.03 | 0.02 | 0.39 |

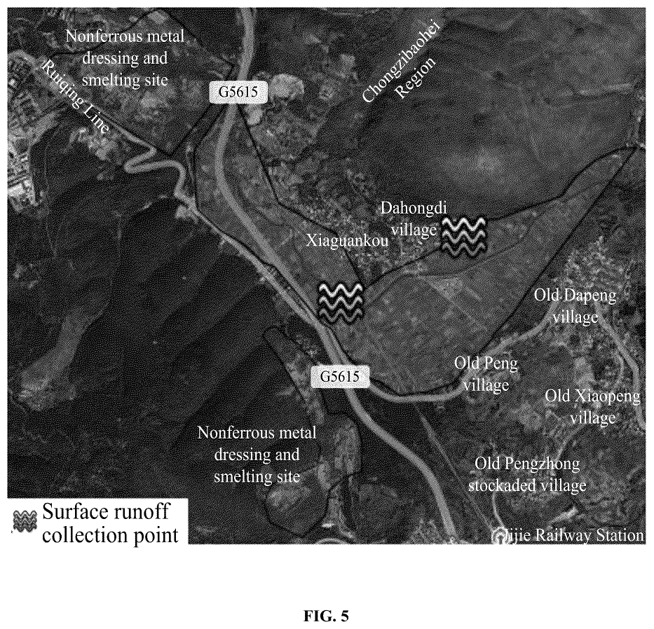

In 2023, according to the reference data obtained from the China Meteorological Data Service Centre-National Meteorological Information Center (cma .cn), the annual average rainfall during the monitoring period (September 2022 to September 2023) is 930 mm; the rainfall during the monitoring period is 1596 mm and an R value is 0.583, corresponding to formula (4). The surface runoff sampling points are as shown in FIG. 5.

The input fluxes of Pb, As, Cu, Cd, and Zn in the monitored region are calculated as 0.07 g/y·ha, 0.13 g/y·ha, 0.03 g/y·ha, 0.02 g/y·ha, and 0.39 g/y·ha, respectively.

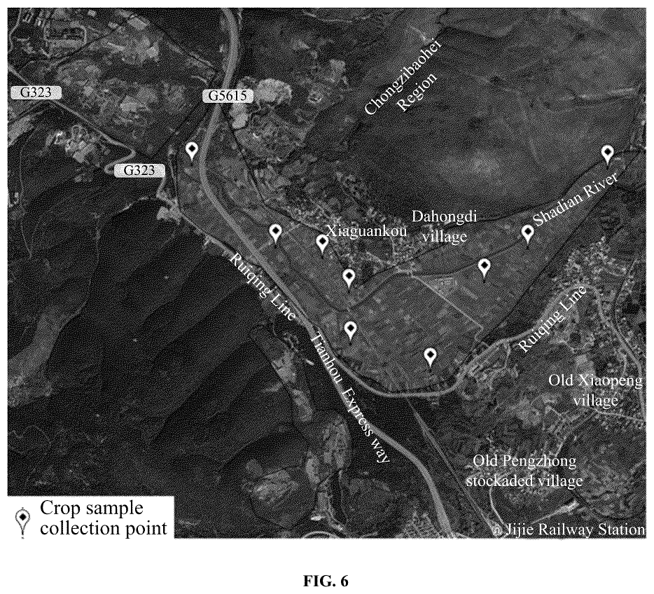

The crop sampling points are as shown in FIG. 6. With reference to formula (5), the output fluxes of the heavy metals through the corn crop are calculated, and results are as shown in Table 2-5.

TABLE 2-5
Crop Output Flux

| Input Flux (g/y · ha) | Pb | As | Cu | Cd | Zn |
|---|---|---|---|---|---|
| Crop output | 46.62 | 83.58 | 20.73 | 15.22 | 260.58 |

### Example 3

An input-output flux inventory is established.

TABLE 3-1
Input-Output Flux Inventory

| | Element | Pb | As | Cu | Cd | Zn |
|---|---|---|---|---|---|---|
| Input | Atmospheric | 226.43 | 262.74 | 77.51 | 28.06 | 802.00 |
| Flux | dust fall | | | | | |
| | Pesticide and | 0.07 | 0.04 | 0.71 | 0.00 | 0.29 |
| | fertilizer | | | | | |
| | Irrigation water | 0.26 | 2.72 | 0.12 | 0.01 | 0.88 |
| | Total input flux | 226.76 | 265.77 | 78.42 | 28.10 | 803.17 |
| | (g/y · ha) | | | | | |
| Input | Surface runoff | 0.07 | 0.13 | 0.03 | 0.02 | 0.39 |
| Flux | water | | | | | |
| | Corn crop | 46.62 | 83.58 | 20.73 | 15.22 | 260.58 |
| | Total input flux | 46.73 | 84.32 | 20.78 | 15.25 | 261.78 |
| | (g/y · ha) | | | | | |

TABLE 3-2
Heavy Metal Input-Output Flux Equilibrium

| Element | Pb | As | Cu | Cd | Zn |
|---|---|---|---|---|---|
| Total input flux | 226.76 | 265.77 | 78.42 | 28.10 | 803.17 |
| (g/y · ha) | | | | | |
| Total output flux | 46.73 | 84.32 | 20.78 | 15.25 | 261.78 |
| (g/y · ha) | | | | | |
| Heavy metal | 180.03 | 181.45 | 57.64 | 12.85 | 541.39 |
| accumulation flux | | | | | |
| (g/y · ha) | | | | | |

To sum up, the accumulated fluxes of Pb, As, Cu, Cd, and Zn in the monitored region in 2023 are calculated according to the emission inventory as 180.03 g/y·ha, 181.45 g/y·ha, 57.64 g/y·ha, 12.85 g/y·ha, and 541.39 g/y·ha, respectively (a negative value indicates that the heavy metals in the element are in a purified state in the year); the average soil density is 1.54 g/cm³; the soil of 0-100 cm is taken as a measurement object; and the accumulated fluxes of the heavy metals Pb, As, Cu, Cd, and Zn in soil per unit mass in the monitored region are distributed as 116.93 mg/kg*y, 118.22 mg/kg*y, 37.44 mg/kg*y, 8.35 mg/kg*y, and 352.07 mg/kg*y, respectively.

### Example 4

### Receptor Model

The PMF model is employed to analyze the heavy metal data of surface soil. In this study, 2, 3, 4, 5, 6, and 7 contribution factors are set in the PMF model separately, and run for 20 times in the PMF model. The results show that when the number of factors is 4, the optimal solution for heavy metal concentration resolution is obtained. Residual values of more than 85% of soil samples are between −3 and 3.

Table 4-1 shows residual analysis of basic model operation. A fitting coefficient (R²) for each element is more than 0.9, which indicates that the results of the PMF model are reliable. According to the PMF results, the sources of heavy metals in soil are divided into four factors, namely factor 1, factor 2, factor 3, and factor 4, which account for 18.9%, 53%, 10.5%, and 17.6% of a total contribution, respectively.

TABLE 4-1
Residual Analysis of Basic PMF Model Results

| | | | Intercept | | | Scaled | Normal |
|---|---|---|---|---|---|---|---|
| Element | Category | R² | S.E. | Slope | Slope SE | residuals | residual |
| Pb | Strong | 0.98810 | 5.88786 | 0.98177 | 0.02471 | 10.08796 | Yes |
| AS | Strong | 0.95256 | 40.27603 | 0.91132 | 0.04666 | 62.52064 | Yes |
| Cu | Strong | 0.99777 | −0.32963 | 1.00140 | 0.01085 | 2.61672 | Yes |
| Cd | Strong | 0.99773 | 0.89476 | 0.97447 | 0.01067 | 1.48929 | Yes |
| As | Strong | 0.98051 | −10.50920 | 1.00748 | 0.03259 | 62.10574 | Yes |
| Total | Strong | 1 | −0.58278 | 1.00033 | 0.00020 | 0.73919 | Yes |

The main contribution elements of the factor 1 are Cd (51.4%), As (22.9%), and Zn (20.9%); the main contribution elements of the factor 2 are Zn (59.7%), Pb (44.3%), and Cd (42.6%); the main contribution element of the factor 3 is Pb (38.2%); and the main contribution elements of the factor 4 are Pb (15.4%), Zn (19.4%), As (12.2%), and Cu (41.3%).

For the PMF model analysis, more accurate results can be obtained with more types of elements and more samples. The technical method provides a calculation idea. For other solutions, a reference may be made to the calculation of more elements.

To sum up, according to the method for predicting heavy metal accumulation in soil based on an emission inventory and a receptor model provided in the present application, atmospheric dust fall, irrigation water, a fertilizer, and a pesticide are used as heavy metal input flux sources of farmland soil, and surface runoff and a crop are used as output fluxes. A heavy metal pollutant input-output flux inventory is established to specify a dynamic equilibrium relationship of heavy metal accumulation in soil, and soil samples are collected and monitored continuously, which provides important help for determining a migration equilibrium of heavy metals in a farmland region affected by a nonferrous metal dressing and smelting slag yard and a source of soil heavy metal pollution, thereby providing theoretical guidance for subsequent prevention and accurate control of heavy metals in soil.

The preferred embodiments of the present application disclosed above are only used to help illustrate the present application. The preferred embodiments neither describe all the details in detail, nor limit the present application to the specific implementations described. Obviously, many modifications and changes may be made based on the content of the present specification. In the present specification, these embodiments are selected and specifically described to better explain the principle and practical application of the present application, so that a person skilled in the art can well understand and use the present application. The present application is only limited by the claims and a full scope and equivalents thereof.

What is claimed is:

1. A method for predicting heavy metal accumulation in soil based on an emission inventory and a receptor model, comprising the following steps:
S1: calculating a spatial-temporal change of a heavy metal accumulation flux in surface soil, and establishing a heavy metal pollutant input-output flux inventory with atmospheric dust fall, irrigation water, a pesticide, and a fertilizer as heavy metal input flux sources to farmland soil and surface runoff water and a crop as output fluxes to investigate a dynamic equilibrium relationship of heavy metal accumulation in soil;
S2: attributing heavy metal sources of farmland soil of a region affected by a nonferrous metal dressing and smelting site to the atmospheric dust fall, the irrigation water, the pesticide, and the fertilizer;
wherein heavy metal output pathways of the farmland soil are the surface runoff and the crop; and
a heavy metal input flux and a heavy metal output flux are annual heavy metal input and output masses per unit area in the farmland soil of the region affected by the nonferrous metal dressing and smelting site, respectively, in units of g/y·ha; and
calculating the heavy metal input flux and the heavy metal output flux of a monitored region;
S3: carding an input flux and output flux inventory according to calculation results of step S2, and calculating a heavy metal input-output equilibrium per unit area per year in surface farmland soil of the region affected by the nonferrous metal dressing and smelting site by establishing an inventory, with a calculation formula being as shown in formula (1):
$$Δ soil = \sum inputs - \sum outputs ; \qquad formula ( 1 )$$
wherein in formula (1), Δsoil represents an annual heavy metal variation of soil per unit area; and
Σinputs and Σoutputs represent the heavy metal input flux and the heavy metal output flux of soil per unit area calculated in step S2, respectively, in units of g/y·ha;
S4: according to the mass equilibrium formula (1) in step S3, calculating a heavy metal accumulation flux, and calculating an annual heavy metal accumulation speed per unit area in the farmland soil of the region affected by the nonferrous metal dressing and smelting site by formula (2):
$$DVsoil = \frac{{Δ}_{soil}}{h * ρ soil} * {10}^{3} ; \qquad formula ( 2 )$$
wherein in formula (2), DVₛₒᵢₗ represents a heavy metal accumulation speed in soil per unit mass, in units of mg/y kg;
Δₛₒᵢₗ represents an annual heavy metal accumulation flux in soil per unit area, in units of g/y·ha;
h represents a soil depth of the monitored region, which is 1 m; and
ρₛₒᵢₗ represents an average soil density of the monitored region; and
S5: with a heavy metal content in the surface soil in step S1 as a study object, employing a positive matrix factorization (PMF) model to perform data analysis, extracting a plurality of factors, identifying the plurality of factors as different source categories with marking components, and then calculating specific contributions of different factors to the heavy metal content in the soil by multiple linear regression, with a specific formula being as shown in formula (8):
$${x}_{ij} = {\sum }_{k = 1}^{p} {g}_{ik} {f}_{kj} + {e}_{ij} ; \qquad formula ( 8 )$$
wherein in formula (8), xᵢⱼ represents a concentration of a jth heavy metal in an ith sample, in units of mg/kg;
p represents a number of factors affecting a heavy metal concentration in a sample;
gᵢₖ represents a mass concentration of a kth factor to the ith sample;
fₖⱼ represents a mass concentration of a jth element in the ith sample; and
eᵢⱼ represents a residual of the jth element in the ith sample; and
minimizing an objective function Q by using a weighted least square method, as shown in formula (9), to obtain a model result:
$$Q = {\sum }_{i = 1}^{n} {{\sum }_{j = 1}^{m} [ \frac{{x}_{ij} - {\sum }_{k = 1}^{p} {g}_{ik} {f}_{kj}}{{u}_{ij}} ]}^{2} \qquad formula ( 9 )$$
wherein in formula (9), uᵢⱼ represents an uncertainty value of the jth heavy metal in the ith sample, which is related to laboratory conditions and a test method, and quantized by formula (10):
$${u}_{ij} = \{ \begin{matrix}\frac{5}{6} \times MDL \\ \sqrt{{( δ \times {x}_{ij} )}^{2} + {( 0.5 \times MDL )}^{2}}\end{matrix} Xij \leq MDL , Xij > MDL . \qquad formula ( 10 )$$
wherein in formula (10), & represents a relationship standard deviation of a heavy metal concentration; and MDL represents a method detection limit.

2. The method for predicting heavy metal accumulation in soil based on an emission inventory and a receptor model according to claim 1, wherein ρₛₒᵢₗ is 1540 kg/m³.

3. The method for predicting heavy metal accumulation in soil based on an emission inventory and a receptor model according to claim 1, wherein step S1 comprises sampling and sample treatment methods of various samples; and the various samples comprise soil, irrigation water, surface runoff, atmospheric dust fall, a crop, a fertilizer, and a pesticide.

4. The method for predicting heavy metal accumulation in soil based on an emission inventory and a receptor model according to claim 3, wherein the sampling of the soil comprises using a five-point sampling method, wherein the five-point sampling method refers to collecting samples at four vertexes and a center of a square and then mixing the samples; a sampling depth is 0-20 cm; in an entire monitored region, a distribution of sampling points conforms to the following principle: starting from a nonferrous metal dressing and smelting slag yard, a sampling point is determined in units of 200 meter in a migration path;
the sample treatment method of the soil comprises: air-drying soil samples, and then sieving all the air-dried soil samples using a 2 mm nylon screen, wherein a filter mesh is stored in a polyethylene bottle; a part of each sample is further ground to pass through a 0.15 mm nylon screen; and after digestion, a total heavy metal content is analyzed; and
the digestion comprises the following specific steps: putting 0.1 g of soil sample into a digestion pot, adding 6 mL of nitric acid, using an acid evaporator for pretreatment at 120° C. for 30 min, cooling, supplementing 1 mL of nitric acid, 2 mL of hydrofluoric acid, and 3 mL of hydrochloric acid, and in a microwave instrument, setting a temperature time gradient as follows:
maintaining the temperature at 150° C. for 10 min, at 180° C. for 5 min, and at 200° C. for 25 min;
after the completion of microwaving, evaporating acid in the acid evaporator at 170° C. for 30 min, during which 1 mL of perchloric acid is added; evaporating acid to about 2 mL, cooling and diluting to 25 mL, and then testing a heavy metal concentration using an inductively coupled plasma-mass spectrometer.

5. The method for predicting heavy metal accumulation in soil based on an emission inventory and a receptor model according to claim 3, wherein surface water is divided into river water, irrigation water, and surface runoff water;
a sampling method for the river water comprises: setting sampling points upstream, midstream, and downstream of a river, and collecting river water samples in polyethylene bottles, with a sampling quantity being 500 mL at a time;
a sampling method for the irrigation water comprises: collecting an irrigation water sample in a polyethylene bottle when a farmer performs irrigation, with a sampling quantity being 500 mL at a time;
a sampling method for the surface runoff water comprises: for runoff from a farmland water outlet to a river, collecting a surface runoff water sample in a polyethylene bottle at the farmland water outlet, while detecting and recording a flow rate;
the sample treatment method of the surface water comprises: for each water sample, sealing and shaking well the water sample, and bringing the water sample back to a laboratory for digestion; and
the digestion comprises the following specific steps: putting a water sample into a crucible, adding 6 mL of nitric acid and 2 mL of hydrogen peroxide, putting a cover on the crucible, placing the crucible on an electric heating plate for heating at 120° C. for two hours, removing the cover, continuously heating until about 5 mL of solution remains, diluting, and then testing a heavy metal concentration using the inductively coupled plasma-mass spectrometer.

6. The method for predicting heavy metal accumulation in soil based on an emission inventory and a receptor model according to claim 3, wherein a sampling method for the atmospheric dust fall comprises: starting from a nonferrous metal dressing and smelting slag yard, setting atmospheric dust fall collection points in a migration path, placing a dust collection jar at a position 5 meter above the ground to avoid a suspended matter in soil from affecting the collection of the atmospheric dust fall, and adding 5 mL of glycol to each dust collection jar to avoid a bacteria breeding exogenous material from affecting a sample composition; then adding 2% of HNO₃ to prevent types of elements from changing, wherein dry and wet deposition occurs without separation when collecting a sample; and packaging the sample in a polyethylene bottle and delivering the polyethylene bottle to the laboratory for treatment;
the sample treatment method of the atmospheric dust fall comprises: standing an atmospheric dust fall sample for 2-3 days, centrifuging in a centrifuge at 3500 rpm for 10 min, transferring a supernatant liquid to a glass bottle, and measuring a volume thereof; transferring remaining precipitate to a beaker, drying under a condition of 60° C. to a constant weight, and recording a mass thereof, wherein the supernatant liquid is a wet deposition sample; the precipitate is a dry deposition sample; digesting the treated dry and wet deposition samples and then detecting a total heavy metal content;
a digestion method of the wet deposition sample is the same as the digestion method of the surface water; and the digestion of the dry deposition sample comprises the following specific steps: putting 0.1 g of dry deposition sample into a digestion pot, adding 5 mL of hydrochloric acid, 5 mL of hydrofluoric acid, and 1 mL of hydrogen peroxide, and in a microwave instrument, setting a temperature time gradient as follows: maintaining the temperature at 150° C. for 10 min, at 180° C. for 5 min, and at 200° C. for 25 min; after the completion of microwaving, evaporating acid in the acid evaporator at 170° C. for 30 min, during which 1 mL of perchloric acid is added; evaporating acid to about 2 mL, cooling and diluting to 25 mL, and then testing a heavy metal concentration using the inductively coupled plasma-mass spectrometer.

7. The method for predicting heavy metal accumulation in soil based on an emission inventory and a receptor model according to claim 3, wherein the crop comprises corn.

8. The method for predicting heavy metal accumulation in soil based on an emission inventory and a receptor model according to claim 3, wherein a sampling method for the crop comprises: collecting a whole plant of the corn crop at harvest time, uniformly distributing sampling points in a monitored region, removing soil from a root, splitting the corn plant into three parts: root, stem leaf, and fruit, putting the three parts in sample bags separately, and bringing the sample bags back to the laboratory for treatment;
the sample treatment method of the crop comprises: using tap water to wash off soil and impurities from a collected corn sample first, then repeatedly washing with deionized water, drying in a drying oven under a condition of 105° C. for 2 h, then completely drying at 60° C. for 48 h, and recording a crop mass after drying; grinding the dried sample, and then testing a total heavy metal content after digestion; and
the digestion comprises the following specific steps: putting 0.1 g of sample into a digestion pot, adding 8 mL of nitric acid for soaking overnight, then adding 1 mL of hydrogen peroxide, microwaving after reacting for a while, and setting a temperature time gradient as follows: maintaining the temperature at 150° C. for 10 min and at 190° C. for 25 min; after the completion of microwaving, putting the sample in an acid evaporator for acid evaporation at 170° C. for 30 min until the sample is about 2 mL, cooling and diluting, and then testing a heavy metal concentration using the inductively coupled plasma-mass spectrometer.

9. The method for predicting heavy metal accumulation in soil based on an emission inventory and a receptor model according to claim 3, wherein the sampling method for the fertilizer and the pesticide comprises: at different growth stages of corn, obtaining 6 fertilizer and pesticide samples; storing the fertilizer samples in sealable bags and the pesticide samples in clean polyethylene bottles, and bringing the fertilizer and pesticide samples back to the laboratory for treatment;
the pesticide samples comprise a Spodoptera litura multicapsid nucleopolyhedrovirus sample, a thiamethoxam-lambda-cyhalothrin sample, a nicosulfuron sample, and a nicosulfuron-dicamba-fluroxypyr sample, and the fertilizer samples comprise a urea sample and a compound fertilizer sample;
the sample treatment method of the fertilizer and the pesticide comprises: testing a total heavy metal content after sample digestion; and the digestion comprises the following specific steps: putting 5 mL of sample into a 100 ml beaker, adding 50% of nitric acid for digestion to be almost dry, dissolving with a 5% nitric acid solution and diluting to 50 mL, and then testing a heavy metal concentration using the inductively coupled plasma-mass spectrometer.

10. The method for predicting heavy metal accumulation in soil based on an emission inventory and a receptor model according to claim 1, wherein step S2 specifically comprises accounting according to formulas (3) to (7):
an atmospheric dust fall input flux:
$${I}_{At} = ( {C}_{w} {V}_{w} + {C}_{d} {W}_{d} ) \times 100 / S ; \qquad formula ( 3 )$$
wherein in formula (3), I_{At} represents a heavy metal flux into the farmland soil of the monitored region through the atmospheric dust fall, in units of g/ha·y;
C_{w} and C_{d} represent heavy metal concentrations in wet deposition and dry deposition, in units of ug/L and ug/g, respectively;
V_{w} and W_{d} represent quantities of wet deposition and dry deposition, in units of L and g, respectively;
S represents an area of a sampling bottle opening, in units of cm²; and 100 is a unit conversion coefficient;
a pesticide and fertilizer input flux:
$${I}_{Fer} = {\sum }_{j = 1}^{n} {F}_{i , j} {C}_{i , j} {10}^{- 6} ; \qquad formula ( 4 )$$
wherein in formula (4):
I_{Fer} represents an annual input flux of a heavy metal fertilizer and pesticide per unit area, in units of g/ha y;
Fᵢⱼ represents an actual application rate of the pesticide and the fertilizer, in units of g/y·ha;
Cᵢⱼ represents a content of an element in the fertilizer and the pesticide, in units of mg/kg or ug/mL; and
n represents a number of types of fertilizers applied at a sampling point;
an irrigation water input flux:
$${I}_{Ir} = {VC}_{i} \cdot {10}^{- 6} ; \qquad formula ( 5 )$$
wherein in formula (5):
I_{Ir} represents an input quantity of a heavy metal (i) from the irrigation water, in units of g/y·ha;
V represents an irrigation water application quantity, in units of L/y·ha; and
Cᵢ represents a concentration of the heavy metal (i) in the irrigation water, in units of ug/L;
a surface runoff output flux:
$${O}_{Run} = {C}_{i} VR \times \frac{100}{S} ; \qquad formula ( 6 )$$
wherein in formula (6):
O_{Run} represents an output quantity of a heavy metal (i) in the surface runoff, in units of g/y·ha;
Cᵢ represents a concentration of the heavy metal (i) in the surface runoff, in units of ug/L;
V represents a volume of the surface runoff water, in units of L;
R represents a ratio of annual average precipitation and rainfall during study, which is unitless; and
S represents an area of a sampling bottle neck, in units of cm²; and
a crop output flux:
$${O}_{Crop} = {\sum }_{j = 1}^{n} {N}_{e , j} {C}_{i , e , j} + {\sum }_{j = 1}^{n} {N}_{n , j} {C}_{i , n , j} + {\sum }_{j = 1}^{n} {N}_{p , j} {C}_{i , n , j} ; \qquad formula ( 7 )$$
wherein in formula (7):
O_{Crop} represents an output of a heavy metal (i) in the crop, in units of g/y·ha;
Nₑⱼ represents a quantity of the root part of the annual crop (j);
C_{i,e,j} represents a concentration of the heavy metal (i) in the root part of the crop (j) in units of ug/g;
N_{n,j} represents a content of a stem leaf part (j) of the annual crop, in units of g/y·ha;
C_{i,n,j} represents a concentration of an element (i) in the stem leaf part (j) of the crop, in units of ug/g;
N_{p,j} represents a number of fruits (p) per year, in units of g/y·ha;
C_{i,p,j} represents a concentration of a heavy metal i in the fruits (p) of the crop, in units of ug/g; and
n represents a number of harvested parts.

11. The method for predicting heavy metal accumulation in soil based on an emission inventory and a receptor model according to claim 4, wherein the heavy metal is lead, zinc, arsenic, copper, or cadmium.

12. The method for predicting heavy metal accumulation in soil based on an emission inventory and a receptor model according to claim 5, wherein the heavy metal is lead, zinc, arsenic, copper, or cadmium.

13. The method for predicting heavy metal accumulation in soil based on an emission inventory and a receptor model according to claim 6, wherein the heavy metal is lead, zinc, arsenic, copper, or cadmium.
